(19) United States
(12) Patent Application Publication
(10) Pub. No.: US 2022/0108449 A1
(43) Pub. Date: Apr. 7, 2022

(54) METHOD AND DEVICE FOR NEURAL NETWORK-BASED OPTICAL COHERENCE TOMOGRAPHY (OCT) IMAGE LESION DETECTION, AND MEDIUM

(71) Applicant: Ping An Technology (Shenzhen) Co., Ltd., Shenzhen (CN)

(72) Inventors: Dongyi FAN, Shenzhen (CN); Lilong WANG, Shenzhen (CN); Rui WANG, Shenzhen (CN); Guanzheng WANG, Shenzhen (CN); Chuanfeng LV, Shenzhen (CN)

(73) Assignee: Ping An Technology (Shenzhen) Co., Ltd., Shenzhen (CN)

(21) Appl. No.: 17/551,460

(22) Filed: Dec. 15, 2021

Related U.S. Application Data
(63) Continuation of application No. PCT/CN2020/117779, filed on Sep. 25, 2020.

(30) Foreign Application Priority Data
May 28, 2020 (CN) 202010468697.0

(51) Int. Cl. G06T 7/00; G16H 50/30; G16H 50/20

(52) U.S. Cl. CPC G06T 7/0012; G16H 50/30; G16H 50/20; G06T 2207/10101; G06T 2207/20088; G06T 2207/20081; G06T 2207/30041; G06T 2207/30096; G06T 2207/20084

(57) ABSTRACT

A method and device for neural network-based optical coherence tomography (OCT) image lesion detection, and a medium are provided. The method includes the following. An OCT image is obtained. The OCT image is inputted into a lesion-detection network model. A position, a category score, and a positive score of each lesion box in the OCT image are outputted through the lesion-detection network model. A lesion detection result of the OCT image is obtained according to the position, the category score, and the positive score of each lesion box. The lesion-detection network model includes a category detection branch configured to obtain, for each of the anchor boxes, a position and a category score of the anchor box, and a lesion positive score regression branch configured to obtain, for each of the anchor boxes, a positive score of whether the anchor box belongs to a lesion, to reflect severity of lesion positive.

### CROSS-REFERENCE TO RELATED APPLICATION(S)

This application is a continuation under 35 U.S.C. § 120 of International Application No. PCT/CN2020/117779, filed on Sep. 25, 2020, which claims priority under 37 U.S.C. § 119(a) and/or PCT Article 8 to Chinese Patent Application No. 202010468697.0, filed on May 28, 2020, the disclosures of which are hereby incorporated by reference in their entireties.

### TECHNICAL FIELD

This disclosure relates to the technical field of artificial intelligence, and particularly to a method and device for neural network-based optical coherence tomography (OCT) image lesion detection, an electronic device, and a computer-readable storage medium.

### BACKGROUND

Optical coherence tomography (OCT) is an imaging technique used for an imaging test of fundus diseases, and has characteristics of high resolution, non-contact, and non-invasiveness. Because of unique optical characteristics of an eyeball structure, OCT has been widely used in the field of ophthalmology, especially in fundus disease testing.

The inventor realizes that the existing OCT-based lesion recognition and detection in ophthalmology is generally implemented by extracting features of an OCT image through a deep convolutional neural network model and training a classifier, which however requires a large number of training samples and manual labeling in training of the neural network model. Generally, 20 to 30 OCT images may be obtained by scanning one eye. Although a large number of training samples can be collected at an image level, costs of collecting a large number of samples at an eye level are very high, which leads to difficulties in model training. As a result, accuracy of a result of the ophthalmic OCT image lesion recognition and detection obtained through the trained model is affected.

The Chinese patent (CN110363226A) relates to a method and device for random forest-based ophthalmic disease classification and recognition, and a medium. An OCT image is input into a lesion recognition model to output a probability value of a lesion category recognized. Then probability values of lesion categories corresponding to all OCT images of a single eye are inputted into a random forest classification model to obtain a probability value of whether the eye corresponds to a disease category, so as to obtain a final disease category result. However, some small lesions cannot be effectively recognized, which may lead to problems such as missed detection and false detection.

### SUMMARY

A first aspect of the disclosure provides a method for neural network-based optical coherence tomography (OCT) image lesion detection. The method includes the following. An OCT image is obtained. The OCT image is inputted into a lesion-detection network model. A position of each lesion box, a category score of each lesion box, and a positive score of each lesion box in the OCT image are outputted through the lesion-detection network model. A lesion detection result of the OCT image is obtained according to the position of each lesion box, the category score of each lesion box, and the positive score of each lesion box. The lesion-detection network model includes a feature-extraction network layer configured to extract image features of the OCT image, a proposal-region extraction network layer configured to extract all anchor boxes in the OCT image, a feature pooling network layer configured to perform average-pooling on feature maps corresponding to all anchor boxes such that the feature maps each have a fixed size, a category detection branch configured to obtain, for each of the anchor boxes, a position and a category score of the anchor box, and a lesion positive score regression branch configured to obtain, for each of the anchor boxes, a positive score of whether the anchor box belongs to a lesion.

A second aspect of the disclosure provides an electronic device. The electronic device includes at least one processor and a memory. The memory is communicatively connected with the at least one processor, and stores instructions executed by the at least one processor. The instructions are executed by the at least one processor to cause the at least one processor to execute all or part of the operations of the method in the first aspect of the disclosure.

A third aspect of the disclosure provides a non-transitory computer-readable storage medium. The non-transitory computer-readable storage medium stores computer programs which, when executed by a processor, cause the processor to execute all or part of the operations of the method in the first aspect of the disclosure.

### BRIEF DESCRIPTION OF THE DRAWINGS

FIG. 1 is a schematic flowchart illustrating a method for optical coherence tomography (OCT) image lesion detection provided in an implementation of the disclosure.

FIG. 2 is a schematic block diagram illustrating a device for OCT image lesion detection provided in an implementation of the disclosure.

FIG. 3 is a schematic diagram of an internal structure of an electronic device configured to implement a method for OCT image lesion detection provided in an implementation of the disclosure.

Objectives, functional characteristics, and advantages of the disclosure will be further described with reference to implementations described below and the accompanying drawings.

### DETAILED DESCRIPTION

It should be understood that, implementations described below are merely used to illustrate the disclosure, which should not be construed as limiting of the disclosure.

Technical solutions of the disclosure may be applicable to the technical field of artificial intelligence, block-chain, and/or big data, for example, the technical solutions of the disclosure particularly relate to neural network technologies. Optionally, data involved in the disclosure, such as a score and a lesion detection result, may be stored in a database or a block-chain, which is not limited in the disclosure.

Implementations of the disclosure will be described in detail below.

According to implementations of the disclosure, a method and device for neural network-based optical coherence tomography (OCT) image lesion detection, an electronic device, and a computer-readable storage medium are provided, which can improve accuracy of lesion detection and avoid problems of missed detection and false detection.

According to implementations of the disclosure, a method for neural network-based OCT image lesion detection is provided. The method includes the following. An OCT image is obtained. The OCT image is inputted into a lesion-detection network model. A position of each lesion box, a category score of each lesion box, and a positive score of each lesion box in the OCT image are outputted through the lesion-detection network model. A lesion detection result of the OCT image is obtained according to the position of each lesion box, the category score of each lesion box, and the positive score of each lesion box. The lesion-detection network model includes a feature-extraction network layer configured to extract image features of the OCT image, a proposal-region extraction network layer configured to extract all anchor boxes in the OCT image, a feature pooling network layer configured to perform average-pooling on feature maps corresponding to all anchor boxes such that the feature maps each have a fixed size, a category detection branch configured to obtain, for each of the anchor boxes, a position and a category score of the anchor box, and a lesion positive score regression branch configured to obtain, for each of the anchor boxes, a positive score of whether the anchor box belongs to a lesion.

According to implementations of the disclosure, a device for neural network-based OCT image lesion detection is provided. The device includes an image obtaining module, a lesion-detection module, a result outputting module. The image obtaining module is configured to obtain an OCT image. The lesion-detection module is configured to input the OCT image into a lesion-detection network model, and output a position of each lesion box, a category score of each lesion box, and a positive score of each lesion box in the OCT image through the lesion-detection network model. The result outputting module is configured to obtain a lesion detection result of the OCT image according to the position of each lesion box, the category score of each lesion box, and the positive score of each lesion box. The lesion-detection network model includes a feature-extraction network layer configured to extract image features of the OCT image, a proposal-region extraction network layer configured to extract all anchor boxes in the OCT image, a feature pooling network layer configured to perform average-pooling on feature maps corresponding to all anchor boxes such that the feature maps each have a fixed size, a category detection branch configured to obtain, for each of the anchor boxes, a position and a category score of the anchor box, and a lesion positive score regression branch configured to obtain, for each of the anchor boxes, a positive score of whether the anchor box belongs to a lesion.

According to implementations of the disclosure, an electronic device is provided. The electronic device includes at least one processor and a memory. The memory is communicatively connected with the at least one processor, and stores instructions executed by the at least one processor. The instructions are executed by the at least one processor to cause the at least one processor to carry out the following actions. An OCT image is obtained. The OCT image is inputted into a lesion-detection network model. A position of each lesion box, a category score of each lesion box, and a positive score of each lesion box in the OCT image are outputted through the lesion-detection network model. A lesion detection result of the OCT image is obtained according to the position of each lesion box, the category score of each lesion box, and the positive score of each lesion box. The lesion-detection network model includes a feature-extraction network layer configured to extract image features of the OCT image, a proposal-region extraction network layer configured to extract all anchor boxes in the OCT image, a feature pooling network layer configured to perform average-pooling on feature maps corresponding to all anchor boxes such that the feature maps each have a fixed size, a category detection branch configured to obtain, for each of the anchor boxes, a position and a category score of the anchor box, and a lesion positive score regression branch configured to obtain, for each of the anchor boxes, a positive score of whether the anchor box belongs to a lesion.

According to implementations of the disclosure, a non-transitory computer-readable storage medium is provided. The non-transitory computer-readable storage medium stores computer programs which, when executed by a processor, cause the processor to carry out the following actions. An OCT image is obtained. The OCT image is inputted into a lesion-detection network model. A position of each lesion box, a category score of each lesion box, and a positive score of each lesion box in the OCT image are outputted through the lesion-detection network model. A lesion detection result of the OCT image is obtained according to the position of each lesion box, the category score of each lesion box, and the positive score of each lesion box. The lesion-detection network model includes a feature-extraction network layer configured to extract image features of the OCT image, a proposal-region extraction network layer configured to extract all anchor boxes in the OCT image, a feature pooling network layer configured to perform average-pooling on feature maps corresponding to all anchor boxes such that the feature maps each have a fixed size, a category detection branch configured to obtain, for each of the anchor boxes, a position and a category score of the anchor box, and a lesion positive score regression branch configured to obtain, for each of the anchor boxes, a positive score of whether the anchor box belongs to a lesion.

In the implementation of the disclosure, lesion detection is performed on the OCT image by means of artificial intelligence and a neural network model. In addition, the lesion positive score regression branch is added to the lesion-detection network model, so that the lesion positive score regression branch obtains, for each of the anchor boxes, a positive score of whether the anchor box belongs to a lesion, to reflect severity of lesion positive. As such, the severity of lesion positive is taken into consideration when obtaining the lesion detection result of the OCT image. On the one hand, the lesion positive score regression branch regresses only a lesion positive degree score, which can avoid inter-class competition and effectively recognize small lesions, and thus the problems of false detection and missed detection can be alleviated, thereby improving the accuracy of lesion detection. On the other hand, a specific quantified score of severity of lesion positive can be obtained through the lesion positive score regression branch, which can be used to urgency judgment.

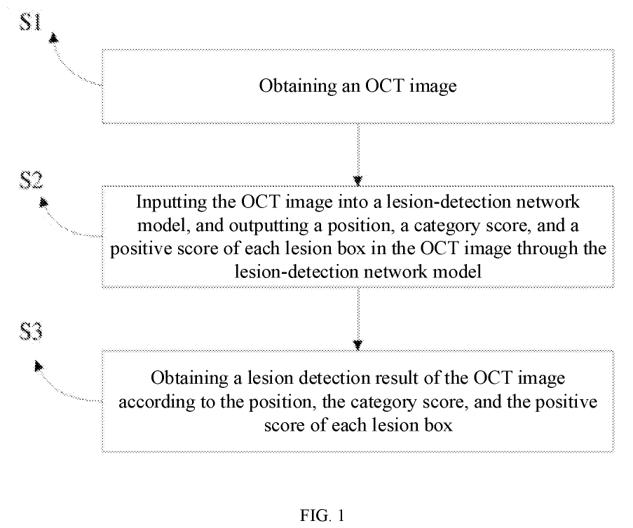

The disclosure provides a method for lesion detection. FIG. 1 is a schematic flowchart illustrating a method for OCT image lesion detection provided in an implementation of the disclosure. The method may be executed by a device, and the device may be software and/or hardware.

In this implementation, the method for neural network-based OCT image lesion detection includes the following. An OCT image is obtained. The OCT image is inputted into a lesion-detection network model. A position, a category score, and a positive score of a lesion box(es) in the OCT image are outputted through the lesion-detection network model. A lesion detection result of the OCT image is obtained according to the position, the category score, and the positive score of the lesion box(es).

The lesion-detection network model herein is a neural network model. The lesion-detection network model includes a feature-extraction network layer, a proposal-region extraction network layer, a feature pooling network layer, a category detection branch, and a lesion positive score regression branch. The feature-extraction network layer is configured to extract image features of the OCT image. The proposal-region extraction network layer, such us a region proposal network (RPN), is configured to extract all anchor boxes in the OCT image. The feature pooling network layer is configured to perform average-pooling on feature maps corresponding to all anchor boxes, such that the feature maps each have a fixed size. The category detection branch is configured to obtain, for each of the anchor boxes, a position and a category score of the anchor box. The lesion positive score regression branch is configured to obtain, for each of the anchor boxes, a positive score of whether the anchor box belongs to a lesion, to reflect severity of lesion positive, which can improve accuracy of the lesion detection result, and can avoid problems of missed detection and false detection due to outputting the lesion detection result based on only the category score.

In one implementation, the feature-extraction network layer includes a feature-extraction layer and an attention mechanism layer. The feature-extraction layer is configured to extract the image features. For example, a ResNet101 network is used to simultaneously extract high-dimensional feature maps at five scales in a form of a pyramid. The attention mechanism layer includes a channel attention mechanism layer and a spatial attention mechanism layer. The channel attention mechanism layer is configured to weight the image features extracted and feature channel weights, so that when the feature-extraction network layer extracts the features, more attention is paid to an effective feature dimension of a lesion. The spatial attention mechanism layer is configured to weight the image features extracted and feature space weights, so that when the feature-extraction network layer extracts the features, the focus is on foreground information rather than background information.

The feature channel weight is obtained as follows. Global max pooling on an a*a*n feature is performed with an a*a convolution kernel, and global average pooling on the a*a*n feature is performed with the a*a convolution kernel, where n represents the number of channels. A result of the global max pooling is added to a result of the global average pooling, to obtain a 1*1*n feature channel weight.

The feature space weight is obtained as follows. Global max pooling on an a*a*n feature is performed with a 1*1 convolution kernel and global average pooling on the a*a*n feature is performed with the 1*1 convolution kernel, to obtain two a*a*1 first feature maps. The two a*a*1 first feature maps are connected in a channel dimension, to obtain an a*a*2 second feature map. A convolution operation is performed on the a*a*2 second feature map (for example, performing the convolution operation on the a*a*2 second feature map with a 7*7*1 convolution kernel), to obtain an a*a*1 feature space weight.

For example, feature maps at five scales extracted by the ResNet101 network include a 128*128*256 feature map, a 64*64*256 feature map, a 32*32*256 feature map, a 16*16*256 feature map, and a 8*8*256 feature map, and feature space weights calculated are different for feature maps of different scales.

In the disclosure, the attention mechanism layer is added to the feature-extraction network layer, so that an attention mechanism is introduced in a feature extraction stage, which can effectively suppress interferences caused by background information, and can extract more effective and robust features for lesion detection and recognition, thereby improving accuracy of lesion detection.

In one implementation, before the feature pooling network layer performs average-pooling on the feature maps corresponding to the anchor boxes, a cropping processing on the feature maps corresponding to the anchor boxes extracted is performed. Specifically, after performing ROI (region of interest) align on features at different scales for cropping to obtain feature maps, average-pooling on the feature maps obtained is performed with a 7*7*256 convolution kernel, such that the feature maps obtained each have a fixed size.

In one implementation, the method further includes the following. After obtaining the OCT image and before inputting the OCT image into the lesion-detection network model, the OCT image is preprocessed. Specifically, the OCT image is preprocessed as follows. Downsampling on the OCT image obtained is performed. The size of an image obtained by downsampling is corrected. As an example, downsampling on an image with an original resolution of 1024*640 is performed to obtain an image with a resolution of 512*320. Then an upper black border and a lower black border are added to obtain a 512*512 OCT image as an input image of the model.

In one implementation, before inputting the OCT image into the lesion-detection network model, the lesion-detection network model is trained.

Further, the lesion-detection network model is trained as follows. An OCT image is collected. The OCT image collected is labeled to obtain a sample image. Taking macula as an example of the lesion for illustration, for each sample image with a macular region scanned through OCT, a location of each lesion box, a category of each lesion box, and severity of each lesion box (including two levels: minor and severe) in the sample image are labeled by at least two doctors. Then each labeling result is reviewed and confirmed by an expert doctor to obtain a final sample-image label, to ensure accuracy and consistency of the labeling. In the disclosure, relatively high sensitivity and specificity can be realized by labeling only a single 2D (two-dimensional) OCT image, which greatly reduces the amount of labeling required and workloads. The sample image labeled is preprocessed. The lesion-detection network model is trained with the sample image preprocessed. A coordinate of the upper left corner, a length, and a width of each lesion box, and a category label of each lesion box labeled in the sample image are used as given values of a model input sample for training. In addition, an enhancement processing (including cropping, scaling, rotation, contrast change, etc.) is performed on the image and a label of the image, to improve a generalization ability of model training. A positive score (where 0.5 represents minor, and 1 represents severe) of each lesion box is used as a training label of the lesion positive score regression branch.

In actual clinical scenarios, doctors generally grade each lesion to judge severity of the lesion instead of directly giving a specific continuous score ranging from 0 to 100, but it is difficult to directly output a label for a lesion between different severity grades through classification. For this reason, in the disclosure, the lesion positive score regression branch performs regression fitting on a given score label (where 0.5 represents minor, and 1 represents severe) instead of direct classification, and therefore, it is more reasonable and effective to perform linear regression on a given grading label value (0.5, 1) to fit a positive score, where the closer an output score is to 1, the more severe the lesion is; and the closer the output score is to 0, the less severe the lesion is or even a false positive.

In one implementation, the lesion detection result of the OCT image is obtained according to the position, the category score, and the positive score of the lesion box(es) as follows. For each anchor box, multiplying a category score of the anchor box and a positive score of the anchor box to obtain a final score of the anchor box. A position and the final score of the anchor box are determined as a lesion detection result of the anchor box. A final lesion detection result can be used to further assist in diagnosis of a disease category corresponding to a macular region of a fundus retina and assist in urgency analysis.

Further, the method further includes the following. Before determining the position and the final score of the anchor box as the lesion detection result of the anchor box, the anchor boxes are merged. As an example, anchor boxes with large overlap are merged through non-maximum suppression. Screening on each anchor box obtained by merging is performed. Specifically, screening is performed according to a category score of each anchor box after merging. For each anchor box obtained by merging, if a category score of the anchor box is greater than or equal to a threshold, the anchor box is assigned as the lesion box; if the category score of the anchor box is less than the threshold, the anchor box is discarded, that is, the anchor box is not assigned as the lesion box. The threshold herein may be set manually, or determined according to a maximum Youden index (i.e., the sum of sensitivity and specificity), where the maximum Youden index may be determined according to a maximum Youden index of a test set during the training of the lesion-detection network model.

In one implementation, the anchor boxes extracted are merged. For each anchor box obtained by merging, the anchor box is assigned as the lesion box on condition that a category score of the anchor box is greater than or equal to a threshold, or the anchor box is discarded on condition that the category score of the anchor box is less than the threshold. For each anchor box assigned as the lesion box: a final score of the anchor box is obtained by multiplying a category score of the anchor box and a positive score of the anchor box, and a position of the anchor box and the final score of the anchor box are determined as a lesion detection result of the anchor box, so as to obtain the lesion detection result of the OCT image.

In the disclosure, in addition to fitting a position of a lesion box and a category score of the lesion box, the lesion positive score regression branch, which is used to reflect severity of lesion positive, is also introduced to quantify severity of a lesion, so as to output a lesion severity score, which is conducive to obtaining an accurate detection result, thereby avoiding problems of missed detection and false detection due to outputting the lesion detection result based on only the category score.

Compared to the existing detection network that outputs only a category score for each target box, on the one hand, when a lesion is similar to two or more categories of lesions in terms of appearance characteristics, a category score obtained through an original detection network is relatively low, so that it is filtered by a threshold. As a result, missed detection occurs. However, in the disclosure, the lesion positive score regression branch regresses on only a lesion positive degree score, which can avoid inter-class competition, thereby alleviating the problems of false detection and missed detection. On the other hand, the lesion-detection network model may detect a small tissue with slight abnormalities but no clinical significance, and determine a relatively high category score for the tissue. In this case, a specific quantified score of severity of lesion positive can also be obtained by the lesion positive score regression branch, which can be used to urgency judgment.

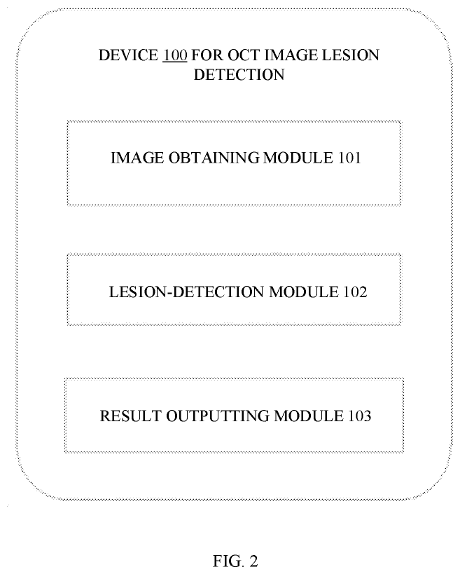

FIG. 2 is a schematic diagram illustrating functional modules of a device for lesion detection provided in the disclosure. A device 100 for OCT image lesion detection of the disclosure may be installed in an electronic device. According to implemented functions, a device for neural network-based OCT image lesion detection may include an image obtaining module 101, a lesion-detection module 102, and a result outputting module 103. The module described in the disclosure can also be called a unit. The module refers to a series of computer program segments that can be executed by a processor of the electronic device and can implement a fixed function, and is stored in a memory of the electronic device.

In this implementation, a function of each module/unit is as follows. The image obtaining module 101 is configured to obtain an OCT image. The lesion-detection module 102 is configured to input the OCT image into a lesion-detection network model, and output a position, a category score, and a positive score of a lesion box(es) in the OCT image through the lesion-detection network model. The result outputting module 103 is configured to obtain a lesion detection result of the OCT image according to the position, the category score, and the positive score of the lesion box(es).

The lesion-detection network model herein includes a feature-extraction network layer, a proposal-region extraction network layer, a feature pooling network layer, a category detection branch, and a lesion positive score regression branch. The feature-extraction network layer is configured to extract image features of the OCT image. The proposal-region extraction network layer is configured to extract all anchor boxes in the OCT image. The feature pooling network layer is configured to perform average-pooling on feature maps corresponding to all anchor boxes, such that the feature maps each have a fixed size. The category detection branch is configured to obtain, for each of the anchor boxes, a position and a category score of the anchor box. The lesion positive score regression branch is configured to obtain, for each of the anchor boxes, a positive score of whether the anchor box belongs to a lesion.

In one implementation, the feature-extraction network layer includes a feature-extraction layer and an attention mechanism layer. The feature-extraction layer is configured to extract the image features. For example, a ResNet101 network is used to simultaneously extract high-dimensional feature maps at five scales in a form of a pyramid. The attention mechanism layer includes a channel attention mechanism layer and a spatial attention mechanism layer. The channel attention mechanism layer is configured to weight the image features extracted and feature channel weights, so that when the feature-extraction network layer extracts the features, more attention is paid to an effective feature dimension of a lesion. The spatial attention mechanism layer is configured to weight the image features extracted and feature space weights, so that when the feature-extraction network layer extracts the features, the focus is on foreground information rather than background information.

The feature channel weight is obtained as follows. Global max pooling on an a*a*n feature is performed with an a*a convolution kernel, and global average pooling on the a*a*n feature is performed with the a*a convolution kernel, where n represents the number of channels. A result of the global max pooling is added to a result of the global average pooling, to obtain a 1*1*n feature channel weight.

The feature space weight is obtained as follows. Global max pooling on an a*a*n feature is performed with a 1*1 convolution kernel and global average pooling on the a*a*n feature is performed with the 1*1 convolution kernel, to obtain two a*a*1 first feature maps. The two a*a*1 first feature maps are connected in a channel dimension, to obtain an a*a*2 second feature map. A convolution operation is performed on the a*a*2 second feature map (for example, performing the convolution operation on the a*a*2 second feature map with a 7*7*1 convolution kernel), to obtain an a*a*1 feature space weight.

In the disclosure, the attention mechanism layer is added to the feature-extraction network layer, so that an attention mechanism is introduced in a feature extraction stage, which can effectively suppress interferences caused by background information, and can extract more effective and robust features for lesion detection and recognition, thereby improving accuracy of lesion detection.

In an implementation, before the feature pooling network layer performs average-pooling on the feature maps corresponding to the anchor boxes, a cropping processing on the feature maps corresponding to the anchor boxes extracted is performed. Specifically, after performing ROI (region of interest) align on features at different scales for cropping to obtain feature maps, average-pooling on the feature maps obtained is performed with a 7*7*256 convolution kernel, such that the feature maps obtained each have a fixed size.

In one implementation, the device for OCT image lesion detection further includes a preprocessing module. The preprocessing module is configured to preprocess the OCT image after obtaining the OCT image and before inputting the OCT image into the lesion-detection network model. Specifically, the preprocessing module includes a downsampling unit and a correction unit. The downsampling unit is configured to perform downsampling on the OCT image obtained. The correction unit is configured to correct the size of an image subjected to downsampling. As an example, downsampling on an image with an original resolution of 1024*640 is performed to obtain an image with a resolution of 512*320. Then an upper black border and a lower black border are added to obtain a 512*512 OCT image as an input image of the model.

In one implementation, the device for OCT image lesion detection further includes a training module. The training module is configured to train the lesion-detection network model.

Further, the lesion-detection network model is trained as follows. An OCT image is collected. The OCT image collected is labeled to obtain a sample image. Taking macula as an example of the lesion for illustration, for each sample image with a macular region scanned through OCT, a location of each lesion box, a category of each lesion box, and severity of each lesion box (including two levels: minor and severe) in the sample image are labeled by at least two doctors. Then each labeling result is reviewed and confirmed by an expert doctor to obtain a final sample-image label, to ensure accuracy and consistency of the labeling. The sample image labeled is preprocessed. The lesion-detection network model is trained with the sample image preprocessed. A coordinate of the upper left corner, a length, and a width of each lesion box, and a category label of each lesion box labeled in the sample image are used as given values of a model input sample for training. In addition, an enhancement processing (including cropping, scaling, rotation, contrast change, etc.) is performed on the image and a label of the image, to improve a generalization ability of model training. A positive score (where 0.5 represents minor, and 1 represents severe) of each lesion box is used as a training label of the lesion positive score regression branch.

In actual clinical scenarios, doctors generally grade each lesion to judge severity of the lesion instead of directly giving a specific continuous score ranging from 0 to 100, but it is difficult to directly output a label for a lesion between different severity grades through classification. For this reason, in the disclosure, the lesion positive score regression branch performs regression fitting on a given score label (where 0.5 represents minor, and 1 represents severe) instead of direct classification, and therefore, it is more reasonable and effective to perform linear regression on a given grading label value (0.5, 1) to fit a positive score, where the closer an output score is to 1, the more severe the lesion is; and the closer the output score is to 0, the less severe the lesion is or even a false positive.

In one implementation, the result outputting module configured to obtain the lesion detection result is configured to: multiply, for each anchor box, a category score of the anchor box and a positive score of the anchor box to obtain a final score of the anchor box; and determine a position and the final score of the anchor box as a lesion detection result of the anchor box. A final lesion detection result can be used to further assist in diagnosis of a disease category corresponding to a macular region of a fundus retina and assist in urgency analysis.

Further, the result outputting module is further configured to merge the anchor boxes, before determining the position and the final score of the anchor box as the lesion detection result of the anchor box. As an example, anchor boxes with large overlap are merged through non-maximum suppression. Screening on each anchor box obtained by merging is performed. Specifically, screening is performed according to a category score of each anchor box after merging. For each anchor box obtained by merging, if a category score of the anchor box is greater than or equal to a threshold, the anchor box is assigned as the lesion box; if the category score of the anchor box is less than the threshold, the anchor box is discarded, that is, the anchor box is not assigned as the lesion box. The threshold herein may be set manually, or determined according to a maximum Youden index (i.e., the sum of sensitivity and specificity), where the maximum Youden index may be determined according to a maximum Youden index of a test set during the training of the lesion-detection network model.

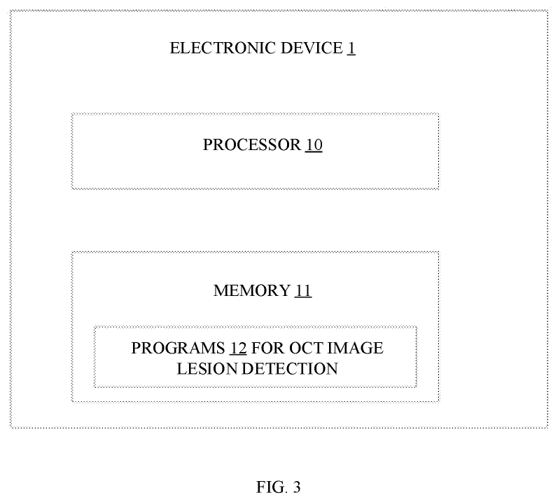

FIG. 3 is a schematic structural diagram illustrating an electronic device configured to implement a method for OCT image lesion detection provided in an implementation of the disclosure. An electronic device 1 may include a processor 10, a memory 11, and a bus. The electronic device 1 may also include computer programs stored in the memory 11 and executed by the processor 10, such as programs 12 for OCT image lesion detection.

The memory 11 at least includes one type of readable storage medium. The readable storage medium may include a flash memory, a mobile hard disk, a multimedia card, a card-type memory (e.g., SD or DX memory, etc.), a magnetic memory, a magnetic disk, an optical disk, and the like. In some implementations, the memory 11 may be an internal storage unit of the electronic device 1, such as a mobile hard disk of the electronic device 1. In other implementations, the memory 11 may also be an external storage device of the electronic device 1, such as a plug-in mobile hard disk equipped on the electronic device 1, a smart media card (SMC), and a secure digital card, a flash card, and so on. Further, the memory 11 may also include both the internal storage unit and the external storage device of the electronic device 1. The memory 11 can not only be used to store application software installed in the electronic device 1 and various data, such as codes of programs for OCT image lesion detection, but also be used to temporarily store data that has been outputted or will be outputted.

In some implementations, the processor 10 may include an integrated circuit(s). As an example, the processor 10 includes a single packaged integrated circuit, or includes multiple integrated circuits with a same function or different functions. The processor 10 may include one or more central processing units (CPU), microprocessors, digital processing chips, graphics processors, and a combination of various control chips, etc. The processor 10 is a control center (control unit) of the electronic device. The processor 10 uses various interfaces and lines to connect the various components of the entire electronic device. The processor 10 runs or executes programs (e.g., programs for OCT image lesion detection) or modules stored in the memory 11, and calls data stored in the memory 11, so as to execute various functions of the electronic device 1 and process data.

The bus may be a peripheral component interconnect (PCI) bus, an extended industry standard architecture (EISA) bus, or the like. The bus may include an address bus, a data bus, a control bus, and so on. The bus is configured to implement a communication connection between the memory 11 and at least one processor 10.

FIG. 3 illustrates an electronic device with components. Those skilled in the art can understand that a structure illustrated in FIG. 2 does not constitute any limitation on the electronic device 1. The electronic device 1 may include more or fewer components than illustrated, or may combine certain components or different components.

As an example, although not illustrated, the electronic device 1 may also include a power supply (e.g., a battery) that supplies power to various components. For instance, the power supply may be logically connected to the at least one processor 10 through a power management device, to enable management of charging, discharging, and power consumption through the power management device. The power supply may also include one or more direct current (DC) power supplies or alternating current (AC) power supplies, recharging devices, power failure detection circuits, power converters or inverters, power status indicators, and any combination thereof. The electronic device 1 may also include various sensors, a Bluetooth module, a Wi-Fi module, etc., which is not limited in the disclosure.

Further, the electronic device 1 may also include a network interface. Optionally, the network interface may include a wired interface and/or a wireless interface (e.g., a Wi-Fi interface, a Bluetooth interface, etc.), which is generally used to establish a communication connection between the electronic device 1 and other electronic devices.

Optionally, the electronic device 1 may also include a user interface. The user interface may be a display, an input unit (e.g., a keyboard), and so on. Optionally, the user interface may also be a standard wired interface or a standard wireless interface. Optionally, in some implementations, the display may be a light-emitting diode (LED) display, a liquid crystal display, a touch-sensitive liquid crystal display, an organic light-emitting diode (OLED) touch device, etc. The display can also be appropriately called a display screen or a display unit, which is used to display information processed in the electronic device 1 and to display a visualized user interface.

It should be understood that, the foregoing implementations are merely used for illustration, and the scope of the disclosure is not limited by the above-mentioned structure.

The programs 12 for OCT image lesion detection stored in the memory 11 of the electronic device 1 are a combination of multiple instructions. The programs, when executed by the processor 10, are operable to carry out the following actions. An OCT image is obtained. The OCT image is inputted into a lesion-detection network model. A position, a category score, and a positive score of a lesion box(es) in the OCT image are outputted through the lesion-detection network model. A lesion detection result of the OCT image is obtained according to the position, the category score, and the positive score of the lesion box(es).

Specifically, for specific implementations of the instructions executed by the processor 10, reference may be made to description of relevant operations of the foregoing implementations described with reference to FIG. 1, which will not be repeated herein.

Further, integrated module/unit of the electronic device 1 may be stored in a computer-readable storage medium when it is implemented in the form of a software functional unit and is sold or used as an independent product. The computer-readable storage medium may include any entity or device capable of carrying computer program codes, a recording medium, a universal serial bus (USB), a mobile hard disk, a magnetic disk, an optical disk, a computer memory, a read-only memory (ROM), and so on.

According to implementation of the disclosure, a computer-readable storage medium is further provided. The computer-readable storage medium is configured to store computer programs. The computer programs, when executed by a processor, are operable to implement all or part of the operations of the method in the foregoing implementations, or implement a function of each module/unit of the device in the foregoing implementations, which will not be repeated herein. Optionally, the medium of the disclosure, such as a computer-readable storage medium, is a non-transitory medium or a transitory medium.

It should be understood that, the equipment, device, and method disclosed in implementations of the disclosure may be implemented in other manners. For example, the device implementations described above are merely illustrative; for instance, the division of the unit is only a logical function division and there can be other manners of division during actual implementations.

The modules/units described as separate components may or may not be physically separated, the components illustrated as modules may or may not be physical units, that is, they may be in the same place or may be distributed to multiple network elements. All or part of the modules may be selected according to actual needs to achieve the objectives of the technical solutions of the implementations.

In addition, the functional modules in various implementations of the disclosure may be integrated into one processing unit, or each unit may be physically present, or two or more units may be integrated into one unit. The above-mentioned integrated unit can be implemented in the form of hardware, or implemented in the form of hardware and a software function module.

Obviously, the disclosure is not limited to the details of the foregoing exemplary implementations. For those skilled in the art, the application can be implemented in other specific forms without departing from the spirit or basic characteristics of the disclosure.

Therefore, no matter from which point of view, the foregoing implementations should be regarded as exemplary and non-limiting. The scope of the disclosure is defined by the appended claims rather than the above description, and therefore, all changes falling within definition and scope of equivalent elements of the claims are included in the disclosure. Any associated reference numbers in the claims should not be regarded as limiting the involved claims.

In addition, it is obvious that the term “including” does not exclude other units or operations/steps, and the singular does not exclude the plural. Multiple units or devices of system claims may also be implemented by one unit or device through software or hardware. The term “second” and the like are used to describe names, rather than describe any specific order.

Finally, it should be noted that, the foregoing implementations are merely used to illustrate the technical solutions of the disclosure and should not be construed as limiting the disclosure. While the disclosure has been described in detail with reference to exemplary implementations, it should be understood by those skill in the art that various changes, modifications, equivalents, and variants may be made to the technical solutions of the disclosure without departing from the spirit and scope of the technical solutions of the disclosure.

What is claimed is:

1. A method for neural network-based optical coherence tomography (OCT) image lesion detection, comprising:
obtaining an OCT image;
inputting the OCT image into a lesion-detection network model, and outputting a position of each lesion box, a category score of each lesion box, and a positive score of each lesion box in the OCT image through the lesion-detection network model; and
obtaining a lesion detection result of the OCT image according to the position of each lesion box, the category score of each lesion box, and the positive score of each lesion box;
the lesion-detection network model comprising:
a feature-extraction network layer, configured to extract image features of the OCT image;
a proposal-region extraction network layer, configured to extract all anchor boxes in the OCT image;
a feature pooling network layer, configured to perform average-pooling on feature maps corresponding to all anchor boxes such that the feature maps each have a fixed size;
a category detection branch, configured to obtain, for each of the anchor boxes, a position and a category score of the anchor box; and
a lesion positive score regression branch, configured to obtain, for each of the anchor boxes, a positive score of whether the anchor box belongs to a lesion.

2. The method for neural network-based OCT image lesion detection of claim 1, wherein the feature-extraction network layer comprises:
a feature-extraction layer, configured to extract the image features; and
an attention mechanism layer comprising:
a channel attention mechanism layer, configured to weight the extracted image features and feature channel weights; and
a spatial attention mechanism layer, configured to weight the extracted image features and feature space weights.

3. The method for neural network-based OCT image lesion detection of claim 2, wherein the feature channel weight is obtained as follows:
performing global max pooling on an a*a*n feature with an a*a convolution kernel, and performing global average pooling on the a*a*n feature with the a*a convolution kernel; and
adding a result of the global max pooling to a result of the global average pooling, to obtain a 1*1*n feature channel weight.

4. The method for neural network-based OCT image lesion detection of claim 2, wherein the feature space weight is obtained as follows:
performing global max pooling on an a*a*n feature with a 1*1 convolution kernel and performing global average pooling on the a*a*n feature with the 1*1 convolution kernel, to obtain two a*a*1 first feature maps;
connecting the two a*a*1 first feature maps in a channel dimension, to obtain an a*a*2 second feature map; and
performing a convolution operation on the a*a*2 second feature map to obtain an a*a*1 feature space weight.

5. The method for neural network-based OCT image lesion detection of claim 1, wherein obtaining the lesion detection result of the OCT image according to the position of each lesion box, the category score of each lesion box, and the positive score of each lesion box comprises:
for each anchor box, multiplying a category score of the anchor box and a positive score of the anchor box to obtain a final score of the anchor box; and
for each anchor box, determining a position of the anchor box and the final score of the anchor box as a lesion detection result of the anchor box, to obtain the lesion detection result of the OCT image.

6. The method for neural network-based OCT image lesion detection of claim 5, further comprising:
before determining, for each anchor box, the position of the anchor box and the final score of the anchor box as the lesion detection result of the anchor box,
merging the anchor boxes; and
for each anchor box obtained by merging:
assigning the anchor box as the lesion box, on condition that a category score of the anchor box is greater than or equal to a threshold; or
discarding the anchor box, on condition that the category score of the anchor box is less than the threshold.

7. The method for neural network-based OCT image lesion detection of claim 1, further comprising:
after obtaining the OCT image and before inputting the OCT image into the lesion-detection network model,
performing downsampling on the OCT image obtained; and
correcting the size of an image obtained by downsampling.

8. The method for neural network-based OCT image lesion detection of claim 1, further comprising:
performing a cropping processing on the feature maps corresponding to the anchor boxes extracted, before the feature pooling network layer performs average-pooling on the feature maps corresponding to the anchor boxes.

9. An electronic device, comprising:
at least one processor; and
a memory, communicatively connected with the at least one processor, and storing instructions executed by the at least one processor;
the instructions are executed by the at least one processor to cause the at least one processor to:
obtain an optical coherence tomography (OCT) image;
input the OCT image into a lesion-detection network model, and output a position of each lesion box, a category score of each lesion box, and a positive score of each lesion box in the OCT image through the lesion-detection network model; and
obtain a lesion detection result of the OCT image according to the position of each lesion box, the category score of each lesion box, and the positive score of each lesion box;
the lesion-detection network model comprising:
a feature-extraction network layer, configured to extract image features of the OCT image;
a proposal-region extraction network layer, configured to extract all anchor boxes in the OCT image;
a feature pooling network layer, configured to perform average-pooling on feature maps corresponding to all anchor boxes such that the feature maps each have a fixed size;
a category detection branch, configured to obtain, for each of the anchor boxes, a position and a category score of the anchor box; and
a lesion positive score regression branch, configured to obtain, for each of the anchor boxes, a positive score of whether the anchor box belongs to a lesion.

10. The electronic device of claim 9, wherein the feature-extraction network layer comprises:
a feature-extraction layer, configured to extract the image features; and
an attention mechanism layer comprising:
a channel attention mechanism layer, configured to weight the extracted image features and feature channel weights; and
a spatial attention mechanism layer, configured to weight the extracted image features and feature space weights.

11. The electronic device of claim 10, wherein the feature channel weight is obtained as follows:
performing global max pooling on an a*a*n feature with an a*a convolution kernel, and performing global average pooling on the a*a*n feature with the a*a convolution kernel; and
adding a result of the global max pooling to a result of the global average pooling, to obtain a 1*1*n feature channel weight.

12. The electronic device of claim 10, wherein the feature space weight is obtained as follows:
performing global max pooling on an a*a*n feature with a 1*1 convolution kernel and performing global average pooling on the a*a*n feature with the 1*1 convolution kernel, to obtain two a*a*1 first feature maps;
connecting the two a*a*1 first feature maps in a channel dimension, to obtain an a*a*2 second feature map; and
performing a convolution operation on the a*a*2 second feature map to obtain an a*a*1 feature space weight.

13. The electronic device of claim 9, wherein the at least one processor configured to obtain the lesion detection result of the OCT image according to the position of each lesion box, the category score of each lesion box, and the positive score of each lesion box is configured to:
for each anchor box, multiply a category score of the anchor box and a positive score of the anchor box to obtain a final score of the anchor box; and
for each anchor box, determine a position of the anchor box and the final score of the anchor box as a lesion detection result of the anchor box, to obtain the lesion detection result of the OCT image.

14. The electronic device of claim 13, wherein the at least one processor is further configured to:
before determining, for each anchor box, the position of the anchor box and the final score of the anchor box as the lesion detection result of the anchor box,
merge the anchor boxes; and
for each anchor box obtained by merging:
assign the anchor box as the lesion box, on condition that a category score of the anchor box is greater than or equal to a threshold; or
discard the anchor box, on condition that the category score of the anchor box is less than the threshold.

15. A non-transitory computer-readable storage medium, storing computer programs which, when executed by a processor, cause the processor to carry out the following actions:
obtaining an optical coherence tomography (OCT) image;
inputting the OCT image into a lesion-detection network model, and outputting a position of each lesion box, a category score of each lesion box, and a positive score of each lesion box in the OCT image through the lesion-detection network model; and
obtaining a lesion detection result of the OCT image according to the position of each lesion box, the category score of each lesion box, and the positive score of each lesion box;
the lesion-detection network model comprising:
a feature-extraction network layer, configured to extract image features of the OCT image;
a proposal-region extraction network layer, configured to extract all anchor boxes in the OCT image;
a feature pooling network layer, configured to perform average-pooling on feature maps corresponding to all anchor boxes such that the feature maps each have a fixed size;
a category detection branch, configured to obtain, for each of the anchor boxes, a position and a category score of the anchor box; and
a lesion positive score regression branch, configured to obtain, for each of the anchor boxes, a positive score of whether the anchor box belongs to a lesion.

16. The non-transitory computer-readable storage medium of claim 15, wherein the feature-extraction network layer comprises:
a feature-extraction layer, configured to extract the image features; and
an attention mechanism layer comprising:
a channel attention mechanism layer, configured to weight the extracted image features and feature channel weights; and
a spatial attention mechanism layer, configured to weight the extracted image features and feature space weights.

17. The non-transitory computer-readable storage medium of claim 16, wherein the feature channel weight is obtained as follows:
performing global max pooling on an a*a*n feature with an a*a convolution kernel, and performing global average pooling on the a*a*n feature with the a*a convolution kernel; and
adding a result of the global max pooling to a result of the global average pooling, to obtain a 1*1*n feature channel weight.

18. The non-transitory computer-readable storage medium of claim 16, wherein the feature space weight is obtained as follows:
performing global max pooling on an a*a*n feature with a 1*1 convolution kernel and performing global average pooling on the a*a*n feature with the 1*1 convolution kernel, to obtain two a*a*1 first feature maps;
connecting the two a*a*1 first feature maps in a channel dimension, to obtain an a*a*2 second feature map; and
performing a convolution operation on the a*a*2 second feature map to obtain an a*a*1 feature space weight.

19. The non-transitory computer-readable storage medium of claim 15, wherein the computer programs causing the processor to carry out the actions of obtaining the lesion detection result of the OCT image according to the position of each lesion box, the category score of each lesion box, and the positive score of each lesion box cause the processor to carry out the following actions:
for each anchor box, multiplying a category score of the anchor box and a positive score of the anchor box to obtain a final score of the anchor box; and
for each anchor box, determining a position of the anchor box and the final score of the anchor box as a lesion detection result of the anchor box, to obtain the lesion detection result of the OCT image.

20. The non-transitory computer-readable storage medium of claim 19, wherein the computer programs further cause the processor to carry out the following actions:
before determining, for each anchor box, the position of the anchor box and the final score of the anchor box as the lesion detection result of the anchor box,
merging the anchor boxes; and
for each anchor box obtained by merging:
assigning the anchor box as the lesion box, on condition that a category score of the anchor box is greater than or equal to a threshold; or
discarding the anchor box, on condition that the category score of the anchor box is less than the threshold.
